(19) United States
(12) Patent Application Publication
(10) Pub. No.: US 2025/0054627 A1
(43) Pub. Date: Feb. 13, 2025

(54) METHOD FOR ASSESSING ACUTE KIDNEY INJURY OF INPATIENT

(71) Applicant: China Medical University, Taichung City (TW)

(72) Inventors: Chin-Chi Kuo, Taichung City (TW); Hsiu-Yin Chiang, Taichung City (TW); Hung-Chieh Yeh, Taichung City (TW); Sheng-Ya Lu, Taichung City (TW); Yi-Ching Chang, Taichung City (TW)

(73) Assignee: China Medical University, Taichung City (TW)

(21) Appl. No.: 18/799,046

(22) Filed: Aug. 9, 2024

Related U.S. Application Data
(60) Provisional application No. 63/531,623, filed on Aug. 9, 2023.

(51) Int. Cl. G16H 50/20; G16H 10/60

(52) U.S. Cl. CPC G16H 50/20; G16H 10/60

(57) ABSTRACT

A method for assessing acute kidney injury of inpatient includes the following steps. An electronic medical record data of an inpatient is captured by a processor, and the electronic medical record data includes a baseline basic serum creatinine concentration data. A target serum creatinine concentration data and a minimum post-admission serum creatinine concentration data of the inpatient are captured by the processor. A dynamic-increasing value of serum creatinine concentration is calculated to obtain by the processor based on the target serum creatinine concentration data, the baseline basic serum creatinine concentration data and the minimum post-admission serum creatinine concentration data. The dynamic-increasing value of serum creatinine concentration is analyzed by the processor to assess whether the inpatient is an inpatient suffering the acute kidney injury or not.

### RELATED APPLICATIONS

This application claims priority to U.S. Provisional Application Ser. No. 63/531,623, filed Aug. 9, 2023, which is herein incorporated by reference.

### BACKGROUND

### Technical Field

The present disclosure relates to a medical information analysis method. More particularly, the present disclosure relates to a method for assessing acute kidney injury of inpatient which is for assessing whether an inpatient suffers from the acute kidney injury or not.

### Description of Related Art

In clinical, the acute kidney injury (AKI) is common in inpatients, there are about 3%-18% of the hospitalized adult patients and 50% of the patients admitted to the intensive care unit suffering from the acute kidney injury during hospitalization, and the acute kidney injury is significantly associated with the mortality and the morbidity.

Although the acute kidney injury is potentially preventable and treatable with timely intervention and care, the acute kidney injury thereof is still not immediately recognized in most inpatients. Further, the clinicians usually mistakenly believe that kidney function in AKI can fully recover, leading to improper management of the condition, so that the patients with unrecognized and untreated acute kidney injury often progress to acute kidney disease (AKD) and further to chronic kidney disease (CKD).

Further, in conventional practice, the recognition and the initial management of the acute kidney injury depend on the awareness, the experiments and the judgment of the healthcare providers, and the non-nephrologists may ignore the examination data and be unaware of the occurrence of the acute kidney injury. Furthermore, the conventional clinical detecting system cannot detect and report the occurrence of the acute kidney injury of the inpatient immediately, resulting in the inpatients with the acute kidney injury occurred during the hospitalization not being well treated and cared.

Therefore, how to develop a method for assessing the acute kidney injury and the acute kidney disease, which is automated, standardized, rapid and with high detection accuracy so as to apply to the inpatients, is a technical issue with clinical application value.

### SUMMARY

The present disclosure provides a method for assessing acute kidney injury of inpatient including the following steps. An electronic medical record data of an inpatient is captured by a processor, and the electronic medical record data includes at least one basic serum creatinine concentration data, wherein the at least one basic serum creatinine concentration data is obtained within an observed time before an admission date of the inpatient, and the at least one basic serum creatinine concentration data includes a baseline basic serum creatinine concentration data. A target serum creatinine concentration data of the inpatient obtained on a target date is captured by the processor, wherein the target date is less than or equal to 7 days after the admission date or greater than 7 days after the admission date. A plurality of post-admission serum creatinine concentration data obtained within a specified period after the admission date of the inpatient are captured by the processor, wherein the plurality of post-admission serum creatinine concentration data include a minimum post-admission serum creatinine concentration data. A dynamic-increasing value of serum creatinine concentration is calculated to obtain by the processor based on the target serum creatinine concentration data and the baseline basic serum creatinine concentration data or based on the target serum creatinine concentration data and the minimum post-admission serum creatinine concentration data. The dynamic-increasing value of serum creatinine concentration is analyzed by the processor to assess whether the inpatient has an acute kidney injury after the admission date.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for assessing acute kidney injury of inpatient according to one example of one embodiment of the present disclosure.

FIG. 2 is a flow chart of a method for assessing acute kidney injury of inpatient according to another example of the one embodiment of the present disclosure.

FIG. 3 is a flow chart of a method for assessing acute kidney injury of inpatient according to further another example of the one embodiment of the present disclosure.

FIG. 4 is a flow chart of a method for assessing acute kidney injury of inpatient according to still another example of the one embodiment of the present disclosure.

FIG. 5 is a flow chart of a method for assessing acute kidney injury of inpatient according to yet another example of the one embodiment of the present disclosure.

### DETAILED DESCRIPTION

The present disclosure will be further exemplified by the following specific embodiments. However, the readers should understand that the present disclosure should not be limited to these practical details thereof, that is, in some embodiments, these practical details are used to describe how to implement the materials and methods of the present disclosure and are not necessary.

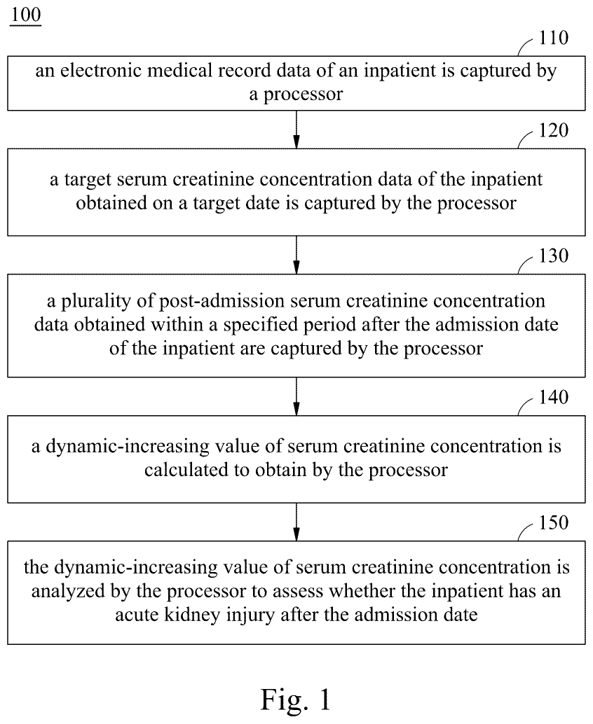

Reference is made to FIG. 1, which is a flow chart of a method 100 for assessing acute kidney injury of inpatient according to one example of one embodiment of the present disclosure. The method 100 for assessing acute kidney injury of inpatient includes Step 110, Step 120, Step 130, Step 140 and Step 150.

In Step 110, an electronic medical record (EMR) data of an inpatient is captured by a processor, and the electronic medical record data includes at least one basic serum creatinine concentration data. The basic serum creatinine concentration data is obtained within an observed time before an admission date of the inpatient, and the basic serum creatinine concentration data includes a baseline basic serum creatinine concentration.

In detail, the electronic medical record data is obtained from all the medical records of examinations and diagnoses of the inpatient at any medical institution before the admission date, and the electronic medical record data can record the diagnosis codes, the blood tests and other related clinical values of the inpatient on a large scale. In the method 100 for assessing acute kidney injury of inpatient of the present disclosure, the basic serum creatinine concentration data is the serum creatinine concentration data of the inpatient obtained within the observed time before the admission date, and the observed time before the admission date can be 7 days to 180 days before the admission date. The selection of time within 7 days to 180 days before the admission date is based on the analysis of current clinical data, wherein the aforementioned time can be used to confirm whether the inpatient had received the nephrotoxic agents before the admission date and to obtain the information of the types of the nephrotoxic drugs and the dose thereof, and it can be used to confirm whether the inpatient had an acute kidney injury event before the admission date and identify the basic kidney function of the inpatient. In other words, the basic serum creatinine concentration data obtained within the aforementioned time may be affected by the nephrotoxic drugs, the inpatient may suffer from the acute kidney disease before the admission date or has the acute kidney injury event or the acute kidney disease after the admission date due to the effects of the nephrotoxic drugs, or the risk of the acute kidney injury of the inpatient after the admission date may be increased. Therefore, the assessing accuracy of the method 100 for assessing acute kidney injury of inpatient of the present disclosure can be enhanced. Furthermore, the electronic medical record data can be stored in the storage devices in the medical institutions or stored in the cloud storage space, but the present disclosure is not limited thereto.

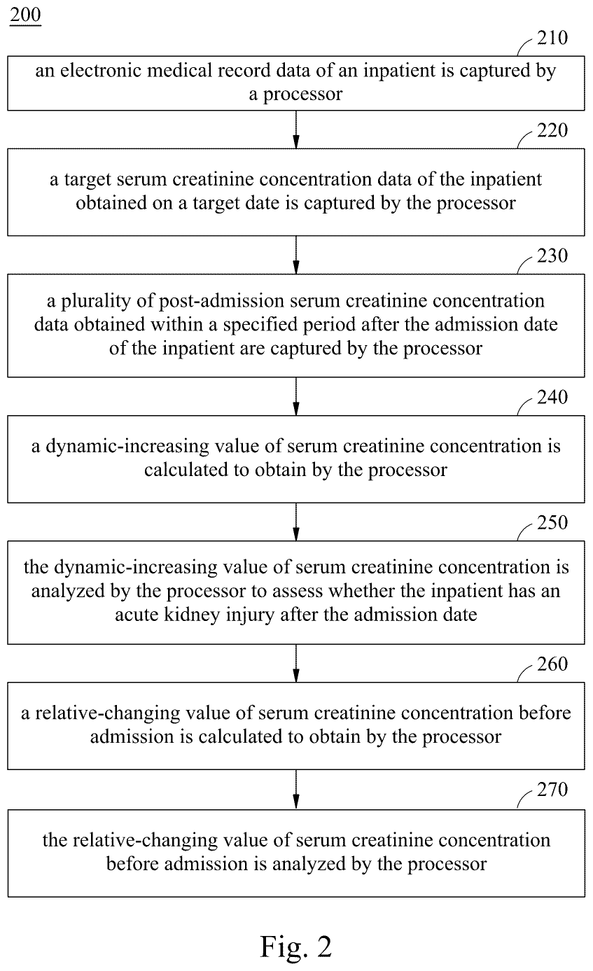

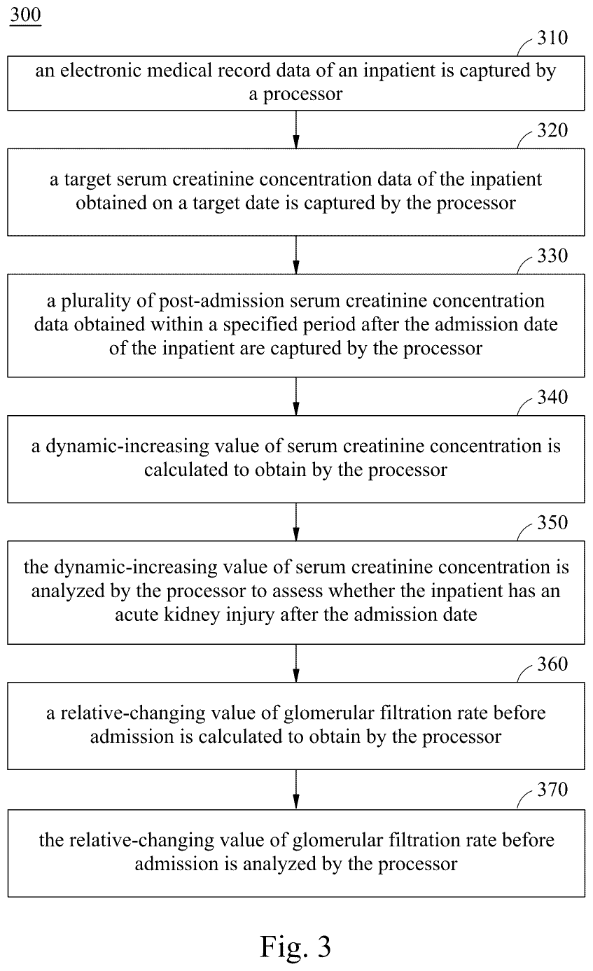

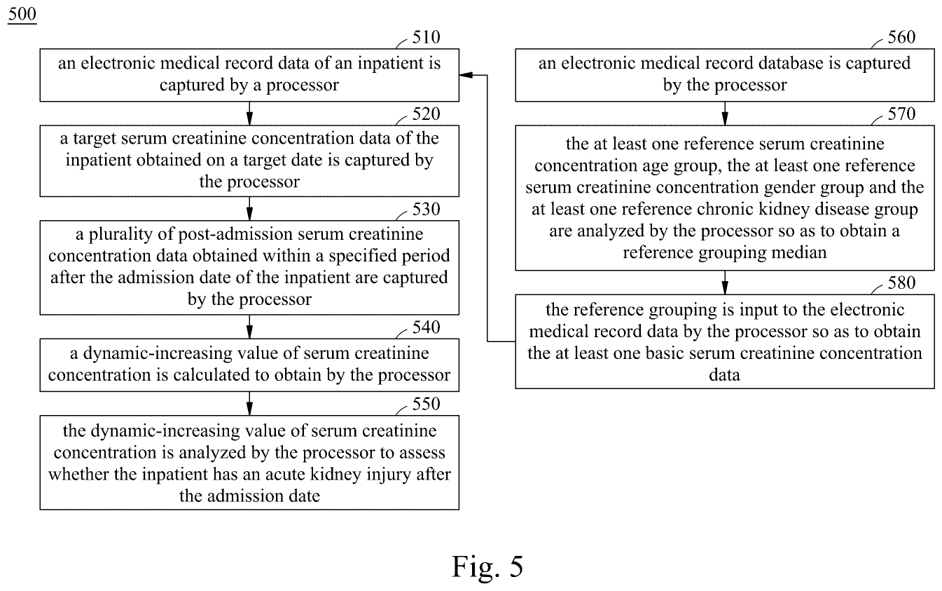

Further, the selection of the baseline basic serum creatinine concentration is performed by the following steps. First, in the method 100 for assessing acute kidney injury of inpatient of the present disclosure, the measurement date of each of the basic serum creatinine concentration data of the inpatient obtained 7 days to 180 days before the admission date is used as a reference, and a minimum among all of the basic serum creatinine concentration data obtained within 180 days before the measurement date is used as the pre-baseline serum creatinine concentration data. However, if there are no records of the basic serum creatinine concentration data within the aforementioned period, the basic serum creatinine concentration data having the minimum value obtained within 365 days before the measurement date is used as the pre-baseline serum creatinine concentration data. Further, if there is also without a record of the basic serum creatinine concentration data obtained within 365 days before the measurement date, the pre-baseline serum creatinine concentration data will be imputed by the method 100 for assessing acute kidney injury of inpatient of the present disclosure based on an imputation method (the details of the imputation method are shown in the method 500 for assessing acute kidney injury of inpatient of FIG. 5). Then, the condition of the acute kidney disease of the inpatient will be defined based on each of the basic serum creatinine concentration data and the pre-baseline serum creatinine concentration data so as to define the baseline basic serum creatinine concentration data thereof. The details of the definition of the acute kidney disease are shown in the description of the data pre-processing before the admission date of the method 200 for assessing acute kidney injury of inpatient and the method 300 for assessing acute kidney injury of inpatient of FIG. 2 and FIG. 3.

Further, if there is without a record of the basic serum creatinine concentration data of the inpatient within the observed time of 7 days to 180 days before the admission date, in the method 100 for assessing acute kidney injury of inpatient of the present disclosure, it is to confirm whether the inpatient has any of the record of the serum creatinine concentration data within 7 days before the admission date. If the inpatient does not have the record of the serum creatinine concentration data within 7 days before the admission date, the baseline basic serum creatinine concentration data will be defined by the imputation method. Further, if the inpatient has the record of the serum creatinine concentration data within 7 days before the admission date, one pre-baseline serum creatinine concentration data of the inpatient will be defined by the imputation method, and then the baseline basic serum creatinine concentration data will be defined based on the condition of the acute kidney disease of the inpatient. In detail, when the inpatient satisfies the criteria of the patient suffering from the acute kidney disease, the baseline basic serum creatinine concentration data of the inpatient will be given based on the imputation method. On the other hand, when the inpatient does not satisfy the criteria of the patient suffering from the acute kidney disease, the record of the serum creatinine concentration data closest to the admission date will be used as the baseline basic serum creatinine concentration data.

Further, if there is only one record of the serum creatinine concentration data of the inpatient within the observed time of 7 days to 180 days before the admission date, the said record of the serum creatinine concentration data will be directly used as the baseline basic serum creatinine concentration data. Furthermore, if there are at least two records of the serum creatinine concentration data of the inpatient within the observed time of 7 days to 180 days before the admission date, the method 100 for assessing acute kidney injury of inpatient of the present disclosure will confirm whether the at least two records of the serum creatinine concentration data are greater than or equal to 4 mg/dL. If at least two records of the serum creatinine concentration data are greater than 4 mg/dL, the serum creatinine concentration data obtained closest to the admission date is used as the baseline basic serum creatinine concentration data. On the other hand, if both the at least two records of the serum creatinine concentration data are not greater than 4 mg/dL, the condition of the acute kidney disease of the inpatient will be assessed based on the aforementioned definition of the acute kidney disease so as to define the baseline basic serum creatinine concentration data thereof.

In more detail, when the inpatient does not satisfy the criteria of the patient suffering from the acute kidney disease, the serum creatinine concentration data obtained closest to the admission date is used as the baseline basic serum creatinine concentration data. On the other hand, if the inpatient satisfies the criteria of the acute kidney disease, the measurement time of the serum creatinine concentration data satisfying the criteria of the acute kidney disease first will be used as the basis. If the acute kidney disease occurs within 90 days before the admission date of the inpatient, it is impossible to assess whether the acute kidney disease of the inpatient is recovered or not, and the baseline basic serum creatinine concentration data will not be defined at this time.

Furthermore, if the acute kidney disease occurs before 90 days before the admission date of the inpatient, the method 100 for assessing acute kidney injury of inpatient of the present disclosure will assess whether the serum creatinine concentration data of the inpatient has declined after the time of the first serum creatinine concentration data satisfying the criteria of the acute kidney disease. If the serum creatinine concentration data of the inpatient has increased by been less than 1.5 times compared with the first serum creatinine concentration data that satisfies the criteria of the acute kidney disease, the acute kidney disease of the inpatient will be considered recovered, and the serum creatinine concentration data obtained closest to the admission date after the decline is used as the baseline basic serum creatinine concentration data. On the other hand, if the serum creatinine concentration data of the inpatient still shows an increase of more than 1.5 times compared with the first serum creatinine concentration data satisfying the criteria of the acute kidney disease, the acute kidney disease of the inpatient will be considered not recovered, and the serum creatinine concentration data obtained closest to the admission date is used as the baseline basic serum creatinine concentration data.

Furthermore, if the serum creatinine concentration data of the inpatient is recorded within 72 hours after dialysis, cardio-pulmonary-cerebral resuscitation or fluid therapy, or is recorded within 24 hours after the massive blood transfusion (8 units of packed red blood cell), the serum creatinine concentration data will not be used as the basic serum creatinine concentration data of the present disclosure so as to prevent the value thereof from being interfered by external factors and lead to inaccurate analysis.

Moreover, the processor used in the method 100 for assessing acute kidney injury of inpatient of the present disclosure can be any general or specific electronic device that can analyze and calculate the basic serum creatinine concentration data, and the processor can include any combination of any type of circuitry with data processing functions. Further, the processor can also be a virtual processor, which can include one or more processors distributed across multiple machines or devices connected by the network, and the present disclosure is not limited thereto. Further, the processor can include the software or the hardware which can be used to perform the data processing, such as a classifier, but the present disclosure is not limited thereto.

In Step 120, a target serum creatinine concentration data of the inpatient obtained on a target date is captured by the processor, wherein the target date is less than or equal to 7 days after the admission date or greater than 7 days after the admission date. In detail, in the method 100 for assessing acute kidney injury of inpatient, the target date can be any day of the inpatient during admission, and the analysis is performed based on the 7 days after the admission date as the watershed. The target serum creatinine concentration data can be any one of the serum creatinine concentration data obtained from the 0^{th} day (the admission date) to the 7^{th} day during admission or any one of the serum creatinine concentration data obtained after the 8^{th} days during admission.

In Step 130, a plurality of post-admission serum creatinine concentration data obtained within a specified period after the admission date of the inpatient are captured by the processor, wherein the plurality of post-admission serum creatinine concentration data include a minimum post-admission serum creatinine concentration data.

In Step 140, a dynamic-increasing value of serum creatinine concentration is calculated to obtain by the processor based on the target serum creatinine concentration data and the baseline basic serum creatinine concentration data or based on the target serum creatinine concentration data and the minimum post-admission serum creatinine concentration data.

In Step 150, the dynamic-increasing value of serum creatinine concentration is analyzed by the processor to assess whether the inpatient has an acute kidney injury after the admission date.

In detail, when the target date is less than or equal to 7 days after the admission date, the dynamic-increasing value of serum creatinine concentration is calculated to obtain according to Formula (I) as follows:

$${SCr}_{dynamic - increasing} = \frac{{SCr}_{target}}{{SCr}_{basic baseline}} , \qquad Formula ( I )$$

wherein SCr_{dynamic-increasing} is the dynamic-increasing value of serum creatinine concentration, SCr_{target} date is the target serum creatinine concentration data, and SCr_{basic baseline} is the baseline basic serum creatinine concentration data. When the dynamic-increasing value of serum creatinine concentration is greater than or equal to 1.5, the inpatient has the acute kidney injury after the admission date. For example, when the target date is 3 days (the 4^{th} day of the admission) after the admission date (the 0^{th} day), the target serum creatinine concentration data is obtained on the 3 days after the admission date, and the dynamic-increasing value of serum creatinine concentration calculated to obtain based on the target serum creatinine concentration data and the baseline basic serum creatinine concentration data will be analyzed by the processor. If the increase of the dynamic-increasing value of serum creatinine concentration is greater than or equal to 1.5, the inpatient satisfies the criteria of the acute kidney disease.

Further, when the target date is less than or equal to 7 days after the admission date or greater than 7 days after the admission date, and the specified period is within 48 hours before the target date, the dynamic-increasing value of serum creatinine concentration is calculated to obtain according to Formula (II) as follows:

$${SCr}_{dynamic - increasing} = {SCr}_{target} - {SCr}_{post - admission minimum} , \qquad Formula ( II )$$

wherein SCr_{dynamic-increasing} is the dynamic-increasing value of serum creatinine concentration, SCr_{target} is the target serum creatinine concentration data, and SCr_{post-admission minimum} is the minimum post-admission serum creatinine concentration. When the dynamic-increasing value of serum creatinine concentration is greater than or equal to 0.3 mg/dL, the inpatient has the acute kidney injury or an acute kidney disease after the admission date. For example, when the target date is 9 days (the 10^{th} day of the admission) after the admission date (the 0^{th} day), the target serum creatinine concentration data is obtained on the 9 days after the admission date. At this time, the specified period falls within the period is the time between the 9 days after the admission date and the 48 hours before the 9 days after the admission date, the plurality of post-admission serum creatinine concentration data are all of the serum creatinine concentration data recorded within the 48 hours, and the minimum post-admission serum creatinine concentration is the minimum of all the serum creatinine concentration data recorded within the 48 hours.

Further, when the target date is greater than 7 days after the admission date, and the specified period is within 7 days before the target date, the dynamic-increasing value of serum creatinine concentration is calculated to obtain according to Formula (III) as follows:

$${SCr}_{dynamic - increasing} = \frac{{SCr}_{target}}{{SCr}_{post - admission minimum}} , \qquad Formula ( III )$$

wherein SCr_{dynamic-increasing} is the dynamic-increasing value of serum creatinine concentration, SCr_{target} is the target serum creatinine concentration data, and SCr_{post-admission minimum} is the minimum post-admission serum creatinine concentration data. When the dynamic-increasing value of serum creatinine concentration is greater than or equal to 1.5, the inpatient has the acute kidney injury or an acute kidney disease after the admission date.

Further, when the dynamic-increasing value of serum creatinine concentration obtained on the target date greater than 7 days after the admission date is greater than or equal to 0.3 mg/dL, or the dynamic-increasing value of serum creatinine concentration is greater than or equal to 1.5, the maximum of the serum creatinine concentration data obtained 7 days before the target date will be compared with the baseline basic serum creatinine concentration data by the method 100 for assessing acute kidney injury of inpatient of the present disclosure. If the value thereof is greater than or equal to 20% of the baseline basic serum creatinine concentration data, it represents that the injury of the kidney slowly increases, and the inpatient is defined as suffering from the acute kidney disease. If the value thereof is less than 20% of the baseline basic serum creatinine concentration data, it represents that the injury of the kidney rapidly increases, and the inpatient is defined as suffering from the acute kidney injury.

Therefore, by analyzing and comparing the target serum creatinine concentration data of the inpatient obtained on the target date after the admission date with the minimum post-admission serum creatinine concentration data obtained within 48 hours before the target date or 7 days before the target date, or comparing with the baseline basic serum creatinine concentration data, it is favorable for monitoring whether the inpatient has developed the acute kidney injury or the acute kidney disease during admission. Thus, it is favorable for adjusting the medical treatment methods for the inpatient immediately, the long-term damage to kidney function caused by the delayed treatment can be avoided, and the method 100 for assessing acute kidney injury of inpatient of the present disclosure has the application potentials in related arts.

Reference is made to FIG. 2, which is a flow chart of a method 200 for assessing acute kidney injury of inpatient according to another example of the one embodiment of the present disclosure. The method 200 for assessing acute kidney injury of inpatient includes Step 210, Step 220, Step 230, Step 240, Step 250, Step 260 and Step 270, wherein Step 210, Step 220, Step 230, Step 240 and Step 250 are the same with Step 110, Step 120, Step 130, Step 140 and Step 150 of FIG. 1, so that the same details are not described again herein.

In the method 200 for assessing acute kidney injury of inpatient, the electronic medical record data can further include a pre-baseline serum creatinine concentration data, and in Step 260, a relative-changing value of serum creatinine concentration before admission is calculated to obtain by the processor based on the pre-baseline serum creatinine concentration data and the at least one basic serum creatinine concentration data. In detail, the relative-changing value of serum creatinine concentration before admission is calculated to obtain according to Formula (IV) as follows:

$${SCr}_{changing} = \frac{{SCr}_{basic}}{{SCr}_{pre - baseline}} , \qquad Formula ( IV )$$

wherein SCr_{changing} is the relative-changing value of serum creatinine concentration before admission, SCr_{basic} is the basic serum creatinine concentration data, and SCr_{pre-baseline} is the pre-baseline serum creatinine concentration data.

In Step 270, the relative-changing value of serum creatinine concentration before admission is analyzed by the processor, wherein when the relative-changing value of serum creatinine concentration before admission is greater than or equal to 1.5, the inpatient is a patient suffering from an acute kidney disease before the admission date.

Therefore, by analyzing the relative-changing value of serum creatinine concentration before admission obtained based on the basic serum creatinine concentration data and the pre-baseline serum creatinine concentration data that are obtained within the observed time before the admission date of the inpatient, it is favorable for confirming whether the inpatient had developed the acute kidney disease before admission. Hence, it is favorable for designing the subsequent medical plans, and the long-term damage to kidney function caused by the delayed treatment can be avoided.

Reference is made to FIG. 3, which is a flow chart of a method 300 for assessing acute kidney injury of inpatient according to further another example of the one embodiment of the present disclosure. The method 300 for assessing acute kidney injury of inpatient includes Step 310, Step 320, Step 330, Step 340, Step 350, Step 360 and Step 370, wherein Step 310, Step 320, Step 330, Step 340 and Step 350 are the same with Step 110, Step 120, Step 130, Step 140 and Step 150 of FIG. 1, so that the same details are not described again herein.

In the method 300 for assessing acute kidney injury of inpatient, the electronic medical record data can further include at least one basic glomerular filtration rate data and a pre-baseline glomerular filtration rate data, and the at least one basic glomerular filtration rate data is obtained within the observed time before the admission date of the inpatient.

In detail, the basic glomerular filtration rate data is obtained from the basic serum creatinine concentration data corresponding thereto according to Formula (V) or Formula (VI) as follows:
Basic glomerular filtration rate data=141×(min(Basic serum creatinine concentration data/0.9, 1)⁻⁴¹¹)×(max(Basic serum creatinine concentration data/0.9, 1)^{-1.209})×(0.993^{age}) Formula (V); or
Basic glomerular filtration rate data=141×(min(Basic serum creatinine concentration data/0.7, 1)^{-0.329})×(max(Basic serum creatinine concentration data/0.7, 1)^{-1.209})×(0.993^{age})×1.018 Formula (VI).

In specific, Formula (V) and Formula (VI) are CKD-EPI (Chronic Kidney Disease Epidemiology Collaboration) equations published by American Society of Nephrology in 2009, and Formula (V) and Formula (VI) can be used to covert the serum creatinine concentration of the patient to obtain the corresponding glomerular filtration rate, wherein Formula (V) is applied to the male subject, and Formula (VI) is applied to the female subject. It must be noted that the CKD-EPI equations used in the present disclosure can be adjusted to the equations published by American Society of Nephrology in different years according to needs, and the present disclosure is not limited thereto. Further, the pre-baseline glomerular filtration rate data is also obtained from the pre-baseline serum creatinine concentration data according to Formula (V) or Formula (VI), and the details thereof will not be described herein.

In Step 360, a relative-changing value of glomerular filtration rate before admission is calculated to obtain by the processor based on the at least one basic glomerular filtration rate data and the pre-baseline glomerular filtration rate data. In detail, the relative-changing value of glomerular filtration rate before admission is calculated to obtain according to Formula (VII) as follows:

$${eGFR}_{changing} = \frac{{eGFR}_{pre - baseline} - {eGFR}_{basic}}{{eGFR}_{pre - baseline}} \times 100 \% , \qquad Formula ( VII )$$

wherein eGFR_{changing} is the relative-changing value of glomerular filtration rate before admission, eGFR_{basic} is the basic glomerular filtration rate data, and eGFR_{pre-baseline} is the pre-baseline glomerular filtration rate data.

In Step 370, the relative-changing value of glomerular filtration rate before admission is analyzed by the processor, wherein when the relative-changing value of glomerular filtration rate before admission is greater than or equal to 35% of the pre-baseline glomerular filtration rate data, the inpatient is a patient suffering from an acute kidney disease before the admission date.

Therefore, by analyzing the relative-changing value of glomerular filtration rate before admission obtained from the basic glomerular filtration rate data and the pre-baseline glomerular filtration rate data that are obtained within the observed time before the admission date of the inpatient, it is favorable for confirming whether the inpatient had developed the acute kidney disease before admission. Hence, it is favorable for designing the subsequent medical plans, and the long-term damage to kidney function caused by the delayed treatment can be avoided.

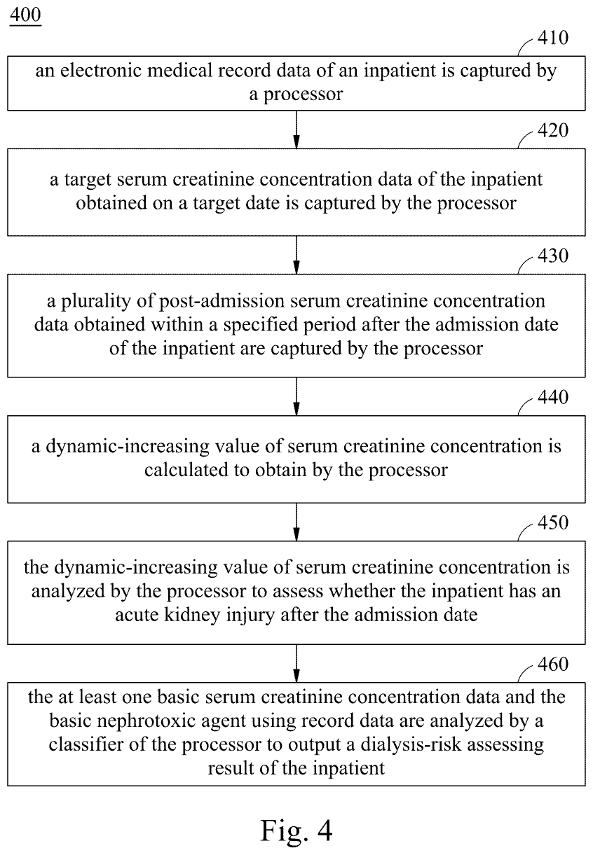

Reference is made to FIG. 4, which is a flow chart of a method 400 for assessing acute kidney injury of inpatient according to still another example of the one embodiment of the present disclosure. The method 400 for assessing acute kidney injury of inpatient includes Step 410, Step 420, Step 430, Step 440, Step 450 and Step 460, wherein Step 410, Step 420, Step 430, Step 440 and Step 450 are the same with Step 110, Step 120, Step 130, Step 140 and Step 150 of FIG. 1, so that the same details are not described again herein.

In the method 400 for assessing acute kidney injury of inpatient, the electronic medical record data can further include a basic nephrotoxic agent using record data, and when the inpatient is the acute kidney injury patient, in Step 460, the at least one basic serum creatinine concentration data and the basic nephrotoxic agent using record data are analyzed by a classifier of the processor to output a dialysis-risk assessing result of the inpatient.

In detail, the plurality of basic nephrotoxic agent using record data are the records of nephrotoxic medication usage before the admission date that may affect the renal function of the inpatient. When the inpatient is assess to have the acute kidney injury after admission, the electronic medical record data will be classified by the classifier in the method 400 for assessing acute kidney injury of inpatient so as to analyze and assess whether the cause of the acute kidney injury in the inpatient is due to the influence of the nephrotoxic drugs. Further, the nephrotoxic drug can be any drug that may affect the renal function and can include non-steroidal anti-inflammatory drugs (NSAIDs), radiocontrast, antimicrobials, chemotherapy and immunotherapy, renin-angiotension system blocker (ARB/ACEi) and diuretics, etc., and the present disclosure is not limited thereto.

Further, the dialysis-risk assessing result can include an acute kidney injury assessing data and a future dialysis-risk matrix, the acute kidney injury assessing data can include an AKI stage assessing result, and the future dialysis-risk matrix can present a predicted dialysis result of the inpatient without receiving the treatment. In detail, the acute kidney injury can be graded by the method 400 for assessing acute kidney injury of inpatient based on the value of the basic serum creatinine concentration data. If the values of the basic serum creatinine concentration data of the inpatient are stable or the change is small, it represents that the acute kidney injury of the inpatient does not rapidly deteriorate. On the contrary, if the values of the basic serum creatinine concentration data of the inpatient have significant changes or a significant increase, the acute kidney injury can be classified as Stage 1, Stage 2 and Stage 3 based on the values thereof. Further, the future dialysis-risk matrix can output a predicted dialysis result within 3 years without receiving any treatment of the inpatient based on the basic glomerular filtration rate data so as to provide the nephrologist with reference for the subsequent treatments. The future dialysis-risk matrix and the corresponding acute kidney injury assessed results are shown in Table 1.

TABLE 1

Value of eGFR
Dialysis risk

| AKI condition | >60 | 45~59 | <45 |
|---|---|---|---|
| No | Normal | Low | Medium |
| Stage 1 | Low | Medium | High |
| Stage 2, Stage 3 | Low | Medium | High |

In Table 1, the unit of the value of eGFR is 60 mL/min/1.73 m²; Stage 1 of the AKI condition represents that the dynamic-increasing value of serum creatinine concentration is greater than or equal to 0.3 mg/dL within 48 hours before the target date or is greater than or equal to 1.5 within 7 days before the target date; Stage 2 of the AKI condition represents that the dynamic-increasing value of serum creatinine concentration is greater than or equal to 2.0 within 7 days before the target date; and Stage 3 of the AKI condition represents that the dynamic-increasing value of serum creatinine concentration is greater than or equal to 4.0 mg/dL within 48 hours before the target date or is greater than or equal to 3 within 7 days before the target date. Further, the selection of the target date is the same with the description of the method 100 for assessing acute kidney injury of inpatient of the present disclosure, and it will not be described herein.

Further, the dialysis-risk assessing result can further include a nephrotoxic drug screening result, and nephrotoxic drug screening result can include an information of a candidate nephrotoxic agent. In detail, if the basic nephrotoxic agent using record data includes a record of at least one of the nephrotoxic drugs, the information of nephrotoxic agent will show the name of the nephrotoxic drug, the receiving duration and the dosage thereof so as to provide the nephrologist with reference for the subsequent treatments, so that the long-term damage to kidney function caused by the delayed treatment can be avoided.

Therefore, by the methods that analyzing the basic serum creatinine concentration data, the basic glomerular filtration rate data and the basic nephrotoxic agent using record data of the inpatient by the classifier to assess the condition of the acute kidney injury and then output the predicted dialysis result without receiving any treatment of the inpatient, the method 500 for assessing acute kidney injury of inpatient of the present disclosure has excellent clinical application potential. Further, the classifier can be a Cox regression classifier, and the present disclosure is not limited thereto.

Reference is made to FIG. 5, which is a flow chart of a method 500 for assessing acute kidney injury of inpatient according to yet another example of the one embodiment of the present disclosure. The method 500 for assessing acute kidney injury of inpatient includes Step 510, Step 520, Step 530, Step 540, Step 550, Step 560, Step 570 and Step 580, wherein Step 510, Step 520, Step 530, Step 540 and Step 550 are the same with Step 110, Step 120, Step 130, Step 140 and Step 150 of FIG. 1, so that the same details are not described again herein.

In detail, the method 500 for assessing acute kidney injury of inpatient is applied to the condition that the inpatient is without any basic serum creatinine concentration data recorded within the observed time before the admission date. At this time, the method 500 for assessing acute kidney injury of inpatient will perform an imputation to the basic serum creatinine concentration data based on the hospital medical record database of outpatients so as to obtain the baseline basic serum creatinine concentration for following analysis.

In Step 560, an electronic medical record database is captured by the processor, wherein the hospital medical record database includes a plurality of reference serum creatinine concentration data of a plurality of subjects, and the plurality of reference serum creatinine concentration data are classified by the processor so as to obtain at least one reference serum creatinine concentration age group, at least one reference serum creatinine concentration gender group and at least one reference chronic kidney disease group. In detail, the plurality of reference serum creatinine concentration data of the plurality of subjects will be classified by the processor based on the age, the gender and the condition of chronic kidney disease of the subjects, and the reference serum creatinine concentration data will be classified as the reference serum creatinine concentration age group, the reference serum creatinine concentration gender group and the reference chronic kidney disease group.

In Step 570, the at least one reference serum creatinine concentration age group, the at least one reference serum creatinine concentration gender group and the at least one reference chronic kidney disease group are analyzed by the processor so as to obtain a reference grouping median.

In Step 580, the reference grouping median is input to the electronic medical record data by the processor so as to obtain the at least one basic serum creatinine concentration data. In detail, the reference grouping median will be input to the electronic medical record data so as to be used as the basic serum creatinine concentration data of the electronic medical record data of the inpatient, and then an appropriate median will be selected as the basic serum creatinine concentration data based on the age, the gender and the condition of chronic kidney disease of the inpatient for following analysis.

Therefore, the method 500 for assessing acute kidney injury of inpatient of the present disclosure can effectively define the baseline basic serum creatinine concentration of the inpatient which is without the serum creatinine concentration data recorded during the observed time before the admission date for the following comparing and analyzing, and the method 500 for assessing acute kidney injury of inpatient of the present disclosure has excellent clinical application potential.

### Example

### <Analysis of Validity of the Imputation of the Basic Serum Creatinine Concentration Data>

In the analysis of the validity of the imputation of the basic serum creatinine concentration data in the method for assessing acute kidney injury of inpatient of the present disclosure, the serum creatinine concentration data of 71,773 cancer-free sepsis patients obtained from the outpatient clinics of China Medical University from 2003 to 2019 will be captured by the processor, and after excluding the records of serum creatinine concentration data from patients whose age do not satisfy the criteria (<18 years or >90 years) or the values are invalid, the serum creatinine concentration data of a total of 5,514 sepsis patients are obtained as the validation group. Further, a total of 2,952 patients with sepsis are without the acute kidney injury, and 2,562 patients with sepsis suffer from the acute kidney injury. Then, the serum creatinine concentration data of the 5,514 sepsis patients are grouped by the processor according to the age, the gender and the condition of chronic kidney disease of the patients with sepsis, and the median of each of the groups is calculated and analyzed by the processor to determine whether the patients with sepsis have the acute kidney injury based on the medians.

Furthermore, in the present analysis, the 5,514 serum creatinine concentration data are simultaneously grouped by the processor, wherein the medians of the groups obtained according to the conventional eGFR 75 method are used as the comparative group 1, and the medians of the groups obtained according to the conventional multiple imputation method are used as the comparative group 2. Then, the values of the medians of each of the verification group, the comparative group 1 and the comparative group 2 are analyzed so as to assess the validity of the imputation of the basic serum creatinine concentration data in the method for assessing acute kidney injury of inpatient of the present disclosure.

Reference is made to Table 2, which shows the analyzing results of the imputation of the basic serum creatinine concentration data of the validation group, the comparative group 1 and the comparative group 2.

TABLE 2

| | Validation | Comparative | Comparative |
|---|---|---|---|
| | group | group 1 | group 2 |
| Mean of | −0.02 | −0.17 | 0.34 |
| difference | (−0.96, 0.91) | (−1.36, 1.02) | (−1.13, 1.80) |
| Sensitivity | 0.82 | 0.85 | 0.69 |
| | (0.80, 0.83) | (0.84, 0.86) | (0.68, 0.71) |
| Specificity | 0.86 | 0.78 | 0.92 |
| | (0.85, 0.87) | (0.77, 0.80) | (0.91, 0.93) |
| Positive | 0.83 | 0.77 | 0.88 |
| predicted | (0.82, 0.85) | (0.76, 0.79) | (0.86, 0.89) |
| value | | | |
| Negative | 0.84 | 0.86 | 0.78 |
| predicted | (0.83, 0.86) | (0.84, 0.87) | (0.76, 0.79) |
| value | | | |
| Agreement | 0.84 | 0.81 | 0.81 |
| | (0.83, 0.85) | (0.80, 0.83) | (0.80, 0.82) |
| Kappa statistic | 0.68 | 0.63 | 0.62 |

As shown in Table 2, the Sensitivity, the Specificity, the positive predicted value, the negative predicted value and the agreement of the validation group are good, the values thereof are higher than 0.82, and the value of Kappa statistic is also up to 0.68.

Reference is further made to Table 3 and Table 4, wherein Table 3 shows the matrix of net reclassification index (NRI) between the validation group and the comparative group 1, and Table 4 shows the matrix of net reclassification index (NRI) between the validation group and the comparative group 2.

TABLE 3

| | Patients with AKI | | | | Patients without AKI | | | |
|---|---|---|---|---|---|---|---|---|
| | (n = 2,562) | | | | (n = 2,952) | | | |
| | Validation group | | | | Validation group | | | |
| | | No | | | | No | | |
| Group | | AKI | AKI | Total | | AKI | AKI | Total |
| Comparative | No | 289 | 96 | 385 | No | 2246 | 70 | 2316 |
| group 1 | AKI | | | | AKI | | | |
| | AKI | 181 | 1996 | 2177 | AKI | 290 | 346 | 636 |
| | Total | 470 | 2092 | 2562 | Total | 2536 | 416 | 2952 |

Reclassification Summary

| Reference | Reclassification by | Patients | Patients |
|---|---|---|---|
| group | Validation group | with AKI | without AKI |
| Comparative | Correct | 96 | 290 |
| group 1 | Incorrect | 181 | 70 |
| | Net | −85 | 220 |

| | |
|---|---|
| Additive NRI* | 4.13% (2.39, 5.90) |
| (95% CI) | |
| Absolute NRI* | 2.45% (1.58, 3.34) |
| (95% CI) | |

*Additive NRI = (−85/2562) + (220/2952) = 4.13%
*Absolute NRI = (−85 + 220)/5514 = 2.45%

TABLE 4

| | Patients with AKI | | | | Patients without AKI | | | |
|---|---|---|---|---|---|---|---|---|
| | (n = 2,562) | | | | (n = 2,952) | | | |
| | Validation group | | | | Validation group | | | |
| | | No | | | | No | | |
| Group | | AKI | AKI | Total | | AKI | AKI | Total |
| Comparative | No | 444 | 339 | 783 | No | 2498 | 205 | 2703 |
| group 2 | AKI | | | | AKI | | | |
| | AKI | 26 | 1753 | 2092 | AKI | 38 | 211 | 249 |
| | Total | 470 | 2092 | 2562 | Total | 2536 | 416 | 2952 |

Reclassification Summary

| Reference | Reference | Reference | Reference |
|---|---|---|---|
| group | group | group | group |
| Comparative | Correct | 339 | 38 |
| group 2 | Incorrect | 26 | 205 |
| | Net | 313 | −167 |

| | |
|---|---|
| Additive NRI* | 6.56% (4.85, 8.39) |
| (95% CI) | |
| Absolute NRI* | 2.65% (1.78, 3.56 |
| (95% CI) | |

*Additive NRI = (313/2562) + (−167/2952) = 6.56%
*Absolute NRI = (313 − 167)/5514 = 2.65%

As shown in Table 3, compared with the comparative group 1, the validation group has improved the classification of the patients without AKI. As shown in Table 4, compared with the comparative group 2, the validation group has improved the classification of the patients with AKI. Therefore, it is shown that the method for assessing acute kidney injury of inpatient of the present disclosure has an excellent validity on the imputation of the basic serum creatinine concentration data, and the performance is equivalent to the conventional imputation method and has a better improvement on the classification validity.

Further, in the present analysis, the aforementioned serum creatinine concentration data of 71,773 cancer-free sepsis patients are analyzed to exclude the serum creatinine concentration data with inconsistent age (<18 years old or >90 years old) or inconsistent values, and a total of 10,136 cancer-free sepsis patients have the acute kidney injury. The diagnosis conditions of the 10,136 cancer-free sepsis patients with the acute kidney injury during the admission are analyzed, and the results thereof are shown in Table 5. The definitions of different stages of the AKI condition are referred to the description of future dialysis-risk matrix, and it will not be described herein.

TABLE 5

| | Person with AKI (person) | | | | |
|---|---|---|---|---|---|
| | Overall | Stage 1 | Stage 2 | Stage 3 | p |
| | 10,136 | 3,912 | 2,777 | 3,447 | value |
| Receiving | 1,056 | 102 | 116 | 838 | <0.001 |
| in-hospital | (10.4%) | (2.6%) | (4.2%) | (24.3%) | |
| nephrology | | | | | |
| consultation | | | | | |
| (Person) | | | | | |
| Time from | 2.0 | 13.5 | 11.0 | 2.0 | <0.001 |
| AKI to | (1.0, 8.0) | (4.0, 31.3) | (2.0, 20.3) | (1.0, 5.0) | |
| consultation | | | | | |
| (Day) | | | | | |
| Receiving | 105 | 10 | 3 | 92 | <0.001 |
| pre-AKI | (1.0%) | (0.3%) | (0.1%) | (2.7%) | |
| consultation | | | | | |

(Person)

As shown in Table 5, only 1.0% of the cancer-free sepsis patients with the acute kidney injury receive nephrology consultation before the acute kidney injury event is detected, and it represents that the physicians did not notice 99% of the patients requiring the nephrology care at the time of the acute kidney injury event. Further, only 10.4% of the cancer-free sepsis patients with the acute kidney injury receive the nephrology consultation after the acute kidney injury event is detected, and the consultation is delayed by 2 days (median) after the acute kidney injury event. It represents that the physicians only captured 10.4% of the patients requiring nephrology care. Furthermore, in view of Stage 3 of the acute kidney injury, only 2.7% and 24.3% of the cancer-free sepsis patients with the acute kidney injury receive the nephrology consult before and after the acute kidney injury event is detected, respectively, and the time from the acute kidney injury event to the nephrology care is still delayed by 2 days. Therefore, the method for assessing acute kidney injury of inpatient of the present disclosure has the potential for market application and can solve the current clinical problems in diagnosing of the acute kidney injury.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for assessing acute kidney injury of inpatient, comprising:
capturing an electronic medical record data of an inpatient by a processor, and the electronic medical record data comprising at least one basic serum creatinine concentration data, wherein the at least one basic serum creatinine concentration data is obtained within an observed time before an admission date of the inpatient, and the at least one basic serum creatinine concentration data comprises a baseline basic serum creatinine concentration data;
capturing a target serum creatinine concentration data of the inpatient obtained on a target date by the processor, wherein the target date is less than or equal to 7 days after the admission date or greater than 7 days after the admission date;
capturing a plurality of post-admission serum creatinine concentration data obtained within a specified period after the admission date of the inpatient by the processor, wherein the plurality of post-admission serum creatinine concentration data comprise a minimum post-admission serum creatinine concentration data;
calculating to obtain a dynamic-increasing value of serum creatinine concentration by the processor based on the target serum creatinine concentration data and the baseline basic serum creatinine concentration data or based on the target serum creatinine concentration data and the minimum post-admission serum creatinine concentration data; and
analyzing the dynamic-increasing value of serum creatinine concentration by the processor to assess whether the inpatient has an acute kidney injury after the admission date.

2. The method for assessing acute kidney injury of inpatient of claim 1, wherein when the target date is less than or equal to 7 days after the admission date, the dynamic-increasing value of serum creatinine concentration satisfies the following condition:
$${SCr}_{dynamic - increasing} = \frac{{SCr}_{target}}{{SCr}_{basic baseline}} ,$$
wherein SCr_{dynamic-increasing} is the dynamic-increasing value of serum creatinine concentration, SCr_{target} is the target serum creatinine concentration data, and SCr_{basic baseline} is the baseline basic serum creatinine concentration data;
wherein when the dynamic-increasing value of serum creatinine concentration is greater than or equal to 1.5, the inpatient has the acute kidney injury after the admission date.

3. The method for assessing acute kidney injury of inpatient of claim 1, wherein when the target date is less than or equal to 7 days after the admission date or greater than 7 days after the admission date, and the specified period is within 48 hours before the target date, the dynamic-increasing value of serum creatinine concentration satisfies the following condition:
$${SCr}_{dynamic - increasing} = {SCr}_{target} - {SCr}_{post - admission minimum} ,$$
wherein SCr_{dynamic-increasing} is the dynamic-increasing value of serum creatinine concentration, SCr_{target} is the target serum creatinine concentration data, and SCr_{post-admission minimum} is the minimum post-admission serum creatinine concentration;
wherein when the dynamic-increasing value of serum creatinine concentration is greater than or equal to 0.3 mg/dL, the inpatient has the acute kidney injury or an acute kidney disease after the admission date.

4. The method for assessing acute kidney injury of inpatient of claim 1, wherein when the target date is greater than 7 days after the admission date, and the specified period is within 7 days before the target date, the dynamic-increasing value of serum creatinine concentration satisfies the following condition:
$${SCr}_{dynamic - increasing} = \frac{{SCr}_{target}}{{SCr}_{post - admission minimum}} ,$$
wherein SCr_{dynamic-increasing} is the dynamic-increasing value of serum creatinine concentration, SCr_{target} is the target serum creatinine concentration data, and SCr_{post-admission minimum} is the minimum post-admission serum creatinine concentration;
wherein when the dynamic-increasing value of serum creatinine concentration is greater than or equal to 1.5, the inpatient has the acute kidney injury or an acute kidney disease after the admission date.

5. The method for assessing acute kidney injury of inpatient of claim 1, wherein the observed time before the admission date is 7 days to 180 days before the admission date.

6. The method for assessing acute kidney injury of inpatient of claim 1, wherein the electronic medical record data further comprises a pre-baseline serum creatinine concentration data, and the method for assessing acute kidney injury of inpatient further comprises:
calculating to obtain a relative-changing value of serum creatinine concentration before admission by the processor based on the pre-baseline serum creatinine concentration data and the at least one basic serum creatinine concentration data; and
analyzing the relative-changing value of serum creatinine concentration before admission by the processor, wherein when the relative-changing value of serum creatinine concentration before admission is greater than or equal to 1.5, the inpatient is a patient suffering from an acute kidney disease before the admission date.

7. The method for assessing acute kidney injury of inpatient of claim 6, wherein the relative-changing value of serum creatinine concentration before admission satisfies the following condition:
$${SCr}_{changing} = \frac{{SCr}_{basic}}{{SCr}_{pre - baseline}} ,$$
wherein SCr_{changing} is the relative-changing value of serum creatinine concentration before admission, SCr_{basic} is the at least one basic serum creatinine concentration data, and SCr_{pre-baseline} is the pre-baseline serum creatinine concentration data.

8. The method for assessing acute kidney injury of inpatient of claim 1, wherein the electronic medical record data further comprises at least one basic glomerular filtration rate data and a pre-baseline glomerular filtration rate data, the at least one basic glomerular filtration rate data is obtained within the observed time before the admission date of the inpatient, and the method for assessing acute kidney injury of inpatient further comprises:
calculating to obtain a relative-changing value of glomerular filtration rate before admission by the processor based on the at least one basic glomerular filtration rate data and the pre-baseline glomerular filtration rate data; and
analyzing the relative-changing value of glomerular filtration rate before admission by the processor, wherein when the relative-changing value of glomerular filtration rate before admission is greater than or equal to 35% of the pre-baseline glomerular filtration rate data, the inpatient is a patient suffering from an acute kidney disease before the admission date.

9. The method for assessing acute kidney injury of inpatient of claim 8, wherein the relative-changing value of glomerular filtration rate before admission satisfies the following condition:
$${eGFR}_{changing} = \frac{{eGFR}_{pre - baseline} - {eGFR}_{basic}}{{eGFR}_{pre - baseline}} \times 100 \% ,$$
wherein eGFR_{changing} is the relative-changing value of glomerular filtration rate before admission, eGFR_{basic} is the at least one basic glomerular filtration rate data, and eGFR_{pre-baseline} is the pre-baseline glomerular filtration rate data.

10. The method for assessing acute kidney injury of inpatient of claim 1, wherein the electronic medical record data further comprises a basic nephrotoxic agent using record data, and when the inpatient has the acute kidney injury patient after the admission date, the method for assessing acute kidney injury of inpatient further comprises:
analyzing the at least one basic serum creatinine concentration data and the basic nephrotoxic agent using record data by a classifier of the processor to output a dialysis-risk assessing result of the inpatient;
wherein the dialysis-risk assessing result comprises an acute kidney injury assessing data and a future dialysis-risk matrix, the acute kidney injury assessing data comprises an AKI stage assessing result, and the future dialysis-risk matrix presents a predicted dialysis result of the inpatient without receiving a treatment.

11. The method for assessing acute kidney injury of inpatient of claim 10, wherein the classifier is a Cox regression classifier.

12. The method for assessing acute kidney injury of inpatient of claim 10, wherein the dialysis-risk assessing result further comprises a nephrotoxic drug screening result, and the nephrotoxic drug screening result comprises an information of a candidate nephrotoxic agent.

13. The method for assessing acute kidney injury of inpatient of claim 1, further comprising:
capturing a hospital medical record database by the processor, wherein the hospital medical record database comprises a plurality of reference serum creatinine concentration data of a plurality of subjects, and the plurality of reference serum creatinine concentration data are classified by the processor so as to obtain at least one reference serum creatinine concentration age group, at least one reference serum creatinine concentration gender group and at least one reference chronic kidney disease group;
analyzing the at least one reference serum creatinine concentration age group, the at least one reference serum creatinine concentration gender group and the at least one reference chronic kidney disease group by the processor so as to obtain a reference grouping median; and
inputting the reference grouping median to the electronic medical record data by the processor so as to obtain the at least one basic serum creatinine concentration data.
